(19) United States
(12) Patent Application Publication
(10) Pub. No.: US 2025/0223570 A1
(43) Pub. Date: Jul. 10, 2025

(54) METHOD FOR PRODUCING INDUCED PLURIPOTENT STEM CELL, NUCLEIC ACID INTRODUCTION CARRIER AND KIT

(71) Applicants: KABUSHIKI KAISHA TOSHIBA, Tokyo (JP); CiRA Foundation, Kyoto-shi (JP)

(72) Inventors: Emi NOZAKI, Shinagawa (JP); Mitsuko ISHIHARA, Setagaya (JP); Masafumi UMEKAGE, Kyoto-shi (JP); Noriko SHIMASAKI, Kyoto-shi (JP)

(73) Assignees: KABUSHIKI KAISHA TOSHIBA, Tokyo (JP); CiRA Foundation, Kyoto-shi (JP)

(21) Appl. No.: 19/000,424

(22) Filed: Dec. 23, 2024

(30) Foreign Application Priority Data
Jan. 5, 2024 (JP) 2024-000718

(51) Int. Cl. C12N 5/074; C12N 15/88

(52) U.S. Cl. CPC C12N 5/0696; C12N 15/88; C12N 2506/1307

(57) ABSTRACT

According to one embodiment, a method is for producing an induced pluripotent stem cell. The method includes bringing a first lipid nanoparticle into contact at least once with a fibroblast, followed by bringing a second lipid nanoparticle into contact at least once with the fibroblast. The first lipid nanoparticle and the second lipid nanoparticle each contain a reprogramming factor. The lipid component composition ratios of the first lipid nanoparticle and the second lipid nanoparticle are different from each other.

### CROSS-REFERENCE TO RELATED APPLICATIONS

This application is based upon and claims the benefit of priority from Japanese Patent Application No. 2024-000718, filed Jan. 5, 2024, the entire contents of which are incorporated herein by reference.

### FIELD

Embodiments described herein relate generally to a method for producing induced pluripotent stem cells, a nucleic acid delivery carrier, and a kit.

### BACKGROUND

Induced pluripotent stem cells, generally referred to as iPS cells, are produced using a viral vector by making the vector to express a reprogramming factor in the cells. When using a viral vector, there is a risk that the vector will remain in the infected cell and continue to express the reprogramming factor. There is also a further risk that the virus may remain in the final product. On the other hand, there is also a method that uses lipofection, but it is not easy to stably or homogeneously produce iPS cells when using this method.

### BRIEF DESCRIPTION OF THE DRAWINGS

FIG. 1 is a cross-sectional view showing an example of lipid nanoparticles for use in a method according to the first embodiment.

FIG. 2 is a schematic diagram showing the method according to the first embodiment.

FIG. 3 is image diagram showing a concept of the method according to the first embodiment.

FIG. 4 is a diagram showing experimental results.

FIG. 5 is a diagram showing experimental results.

### DETAILED DESCRIPTION

In general, according to one embodiment, a method is for producing an induced pluripotent stem cell. The method includes bringing a first lipid nanoparticle into contact at least once with a fibroblast, followed by bringing a second lipid nanoparticle into contact at least once with the fibroblast. The first lipid nanoparticle and the second lipid nanoparticle each contain a reprogramming factor. The lipid component composition ratios of the first lipid nanoparticle and the second lipid nanoparticle are different from each other.

Embodiments will now be described with reference to the accompanying drawings. In these embodiments, substantially identical structural elements are denoted by the same symbols, and some of the descriptions may be omitted. Note that the drawings are schematic, and the relationship between the thickness and planar dimensions of each part, the ratio of the thickness of each part, etc., may differ from the actual ones.

### First Embodiment

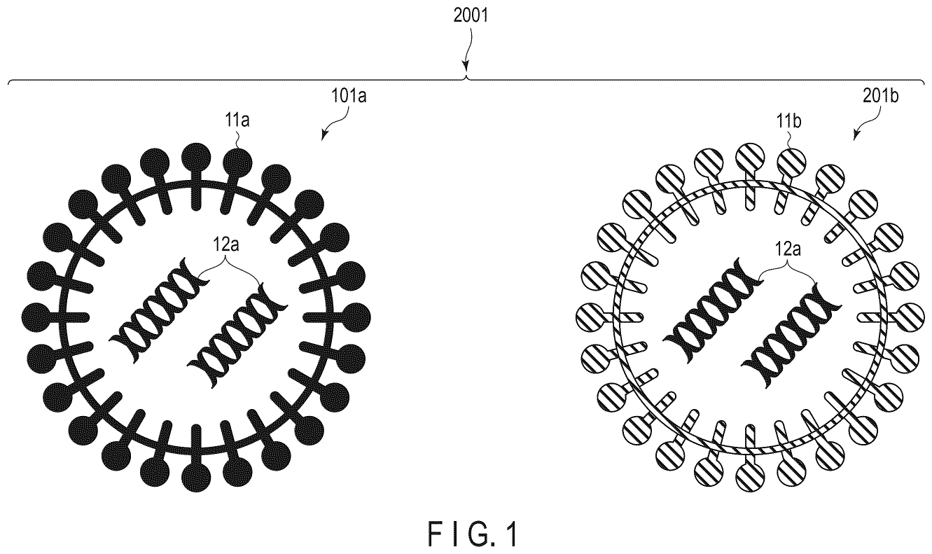

The method according to the embodiment is a method for producing induced pluripotent stem cells using lipid nanoparticles, for example, liposomes. Hereinafter, induced pluripotent stem cells are referred to as iPS cells. First, with reference to FIG. 1, the lipid nanoparticles used in the method of the embodiment are described. An introduction carrier set 2001 shown in FIG. 1 includes a first introduction carrier 101a and a second introduction carrier 201b. The first introduction carrier 101a includes lipid nanoparticles 11a (for example, liposomes 11a) forming a hollow portion and a reprogramming factor 12a encapsulated therein. The second introduction carrier 201b includes lipid nanoparticles 11b (for example, liposomes 11b) forming a hollow portion and a reprogramming factor 12a encapsulated therein. The lipid nanoparticles 11a and 11b form a hollow body made from a lipid membrane, and for example, they are liposomes. The lipid nanoparticles of the second introduction carrier 201b have a lipid component composition ratio different from the lipid component composition ratio of the lipid nanoparticles of the first introduction carrier 101a. The first introduction carrier 101a is, in other words, first lipid nanoparticles that encapsulate a reprogramming factor. The second introduction carrier 201b is, in other words, second lipid nanoparticles that encapsulate a reprogramming factor.

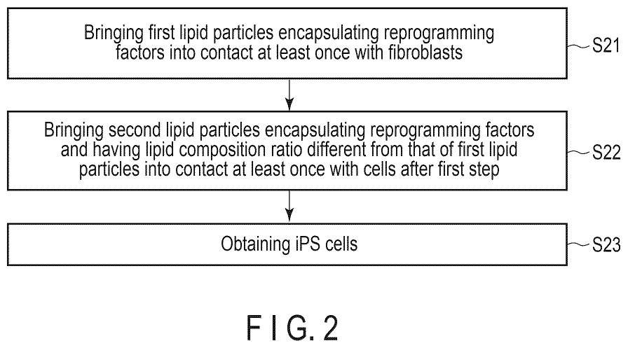

The method includes, as shown in FIG. 2, bringing the first lipid nanoparticles into contact at least once with fibroblasts (S21), followed by bringing the second lipid nanoparticle into contact at least once with the fibroblasts (S22), and obtaining iPS cells (S23). Note here that the first lipid nanoparticles and the second lipid nanoparticles contain a reprogramming factor, respectively. Further, the lipid component composition ratio of the first lipid nanoparticles and that of the second lipid nanoparticle are different from each other. With the introduction carrier set 2001 having such a structure as discussed above, more specifically, by repeatedly introducing the reprogramming factor into the fibroblasts using two types of lipid nanoparticles composed of different lipid components, it is possible to prevent the reversion of reprogramming that may occur in the first half stage of reprogramming. In this way, the effect of the reprogramming factor can be maintained and/or enhanced, thereby further making it possible to efficiently produce iPS cells. Furthermore, because such a method uses non-viral materials to produce iPS cells, there is no need to be concerned about infection or virus-derived risks unlike the cases of using viral materials. Further, because production can be made stable, and therefore it is possible to produce iPS cells uniformly. Here, for the obtaining of iPS cells (S23), it is sufficient if the fibroblasts are substantially transferred to iPS cells through the contact, and it may or may not involve the physical collection of iPS cells.

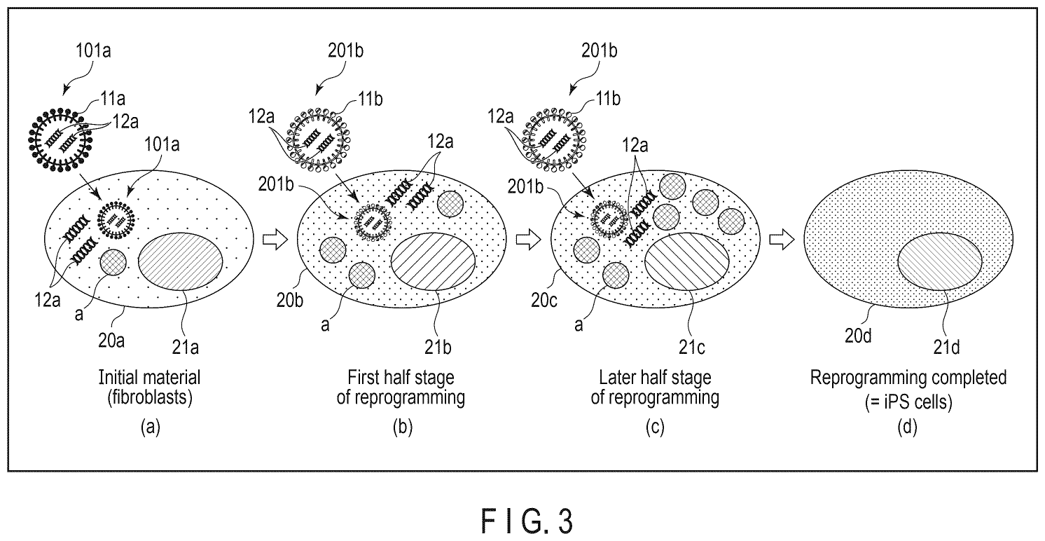

A concept of the method will be further explained with reference to FIG. 3. FIG. 3 shows images of the procedure along a progression in the method and also images showing the change in the state of a single fibroblast over time. The first introduction carrier 101a is administered to the initial material, a fibroblast 20a (part (a) of FIG. 3). The first introduction carrier 101a comes into contact with the initial material, a fibroblast 20a, and releases the reprogramming factor 12a encapsulated in the lipid nanoparticles 11a into the cytoplasm (part (a) of FIG. 3). The reprogramming factor 12a thus released forms a reprogramming substance a in the cytoplasm (part (a) of FIG. 3). By the reprogramming substance a, the information of the nucleus 21a of the fibroblast 20a is gradually reprogrammed, and the state of the cell is shifted to the first half stage of reprogramming (part (a) of FIG. 3→part (b) of FIG. 3). During this first half stage of reprogramming, the second introduction carrier 201b is administered (part (b) of FIG. 3). The second introduction carrier 201b thus administered comes into contact with the fibroblast 20b in the state of the first half stage of reprogramming, and releases the reprogramming factor 12a encapsulated in the lipid nanoparticles 11b into the cytoplasm (part (b) of FIG. 3). The reprogramming factor 12a thus released forms a further reprogramming substance a in the cytoplasm (part (b) of FIG. 3). By the reprogramming substance a, the information of the nucleus 21b of the fibroblast 20b is gradually reprogrammed and the state of the cell is shifted to the latter half stage of reprogramming (part (b) of FIG. 3→part (c) of FIG. 3). During this latter half stage of reprogramming, the second introduction carrier 201b is re-administered (part (c) of FIG. 3). The second introduction carrier 201b thus administered comes into contact with the fibroblast 20c in the state of the later half stage of the reprogramming process, and releases the reprogramming factor 12a encapsulated in the lipid nanoparticle 11b to the cytoplasm (part (c) of FIG. 3). The reprogramming factor 12a thus released forms a further reprogramming substance a in the cytoplasm (part (c) of FIG. 3). By the reprogramming substance a, the information of the nucleus 21c of the fibroblast 20c is gradually reprogrammed, and the state of the cell is shifted to a state in which the reprogramming is complete (part (c) of FIG. 3→part (d) of FIG. 3). Thus, the reprogramming is completed, and a reprogrammed cell 20d, that is, an iPS cell, is obtained.

As described above, when the reprogramming substance a is added to the cytoplasm, the information of the nucleus 21a of the initial material, fibroblast 20a, is gradually reprogrammed. As a result, the state of the cell changes from the initial material, the fibroblast 20a, to the fibroblast 20b in the state of the first half stage of reprogramming), to the fibroblast 20c in the state of the later half stage of reprogramming), to the reprogrammed cell 20d (iPS cell) (that is, it is shifted) (parts (a), (b), (c), and (d) of FIG. 3). The reprogramming factor 12a is, for example, RNA. When a factor necessary for reprogramming is introduced as RNA, the RNA remains in the cytoplasm and is translated into a protein. The protein that is translated is, for example, a transcription factor. With the transcription factor, the genetic information in the nucleus is reprogramed. Incidentally, the introduced RNA is degraded in the cytoplasm within about 24 to 48 hours after introduction. The cellular changes that occur within about 24 to 48 hours after introduction correspond to the first half stage of the reprogramming process. When the second half stage of the reprogramming process is included, the time required for the initial material, the fibroblast, to complete reprogramming and become a reprogrammed cell (that is, iPS cell) is about 96 hours (about 4 days) after the introduction. Here, not that in particular, in the first half stage of the reprogramming process, a phenomenon called “incomplete reprogramming” is likely to occur.

The process from the start of the method to the first half stage of the reprogramming is carried out as shown in parts (a) and (b) of FIG. 3, and the details of the process are as follows:
administering the first introduction carrier 101a to the initial material, fibroblast 20a;
the first introduction carrier 101a coming into contact with the fibroblast 20a;
the first introduction carrier 101a releasing the reprogramming factor 12a encapsulated in the lipid nanoparticles 11a into the cytoplasm;
the released reprogramming factor 12a forms the reprogramming substance a in the cytoplasm;
the reprogramming substance a initiating the reprogramming of the information in the nucleus 21a of the fibroblast 20a; and
the state of the fibroblast 20a being shifted to the first half stage of the reprogramming period.

The incomplete reprogramming is a reaction that cancels the reaction that has advanced to the first half stage of the reprogramming period, that is, a reaction that maintains homeostasis. As a result, the reprogramming induced by the first introduction carrier 101a is reset to have never occurred, and the state of the cell returns to the state of the initial material.

The method according to the embodiment is derived from the inventors' attention to the fact that such an incomplete reprogramming occurs even if the administration of the first introduction carrier 101a is continued. That is, by using second lipid nanoparticles having a lipid component composition ratio different from the lipid component composition ratio of the first lipid nanoparticles during the first half stage of the reprogramming process, so as to further bring the reprogramming factor 12a in, the reprogramming process can be efficiently completed and the object cells can be thus obtained.

The first lipid nanoparticles are configured to have a lipid component composition ratio having an affinity appropriate for the state of the initial material. The second lipid nanoparticles are configured to have a lipid component composition ratio having an affinity appropriate for the cells in which the reprogramming is started. By combining the first lipid nanoparticles and the second lipid nanoparticles and administering them sequentially, the reprogramming can be performed efficiently. Thus, it becomes possible to stably obtain reprogrammed cells. Further, it is also possible to obtain homogeneous reprogrammed cells.

The contact between the first lipid nanoparticles and the fibroblasts as the initial material may be, for example, once, twice, three times, four times, once or more, twice or more, three times or more, or four times or more. The contact between the second lipid nanoparticles and the fibroblasts in the state of the first half stage of the reprogramming may be, for example, one time, two times, three times, four times, one or more times, two or more times, three or more times, or four or more times. Hereafter, the contact between the first lipid nanoparticles and the fibroblasts as the initial material may as well be referred to as the first contact. Further, the contact between the second lipid nanoparticles and the fibroblasts in the state of the first half stage of reprogramming may as well be referred to as the second contact. Basically, a group of the first contacts are performed as a previous process before a group of the second contacts. Alternatively, the first contacts may as well be performed before the second contacts. For example, the first one of the second contacts may be carried out after the first one of the first contacts. For example, the first one of the second contacts may be carried out at the same time as any from the second one of the first contacts onwards. For example, any from the second one of the first contacts onwards may be carried out before, at the same time as, or after the first one of the second contacts. For example, the first lipid nanoparticles (for example, liposomes A) may be added on Day 0 and the second lipid nanoparticles (for example, liposomes B) may be added on Days 2 and 4 to produce iPS cells. Although they are not particularly limited to these, for example, the composition of the liposomes A contain FFT-10, FFT-20, DOPE, DOTAP, cholesterol, and DMG-PEG, and the percent molar ratio thereof is 58:0:0:16:24:2, and the lipid composition of the liposomes B may contain FFT-10, FFT-20, DOPE, DOTAP, cholesterol, and DMG-PEG, and the percent molar ratio may be 0:32:5:9:51:3.

The first contact and the second contact can be carried out in an environment suitable for maintaining fibroblasts, that is, the initial material, fibroblasts in which the reprogramming thereof has been initiated, and cells in which the reprogramming of fibroblasts has been completed, such as a culture medium or a physiological aqueous solution. The culture medium or physiological solution may be replaced as necessary. The environment is maintained in an appropriate way for the cells during or after any contact, or until further contact. For example, it may be incubation. The incubation conditions may be general conditions suitable for fibroblasts, that is, for example, 35° C. to 38° C.

The lipid nanoparticles 11a and the lipid nanoparticles 11b are each designed to have affinity for the respective target cell. In other words, the lipid nanoparticles 11a are designed to have an affinity appropriate for target cells in the first state, and the lipid nanoparticles 11b are designed to have an affinity appropriate for target cells in the second state. The target cells in the first state are fibroblasts in the state of the initial material. The target cells in the second state are fibroblasts that have come into contact with the lipid nanoparticles 11a, fibroblasts in which reprogramming has been started, or fibroblasts in which reprogramming has been initiated. That is, they are fibroblasts that are in a state of having been shifted (changed) after the target cells in the first state come into contact with the lipid nanoparticles 11a. For example, the target cells in the second state can be fibroblasts at any point after they come into contact with the lipid nanoparticles 11a. They can be cells that have come into contact with the lipid nanoparticles 11a but have not yet completed the reprogramming reversal. For example, the target cells in the second state may be cells at the time within 24 hours, within 36 hours, or within 48 hours after coming in contact with the lipid nanoparticles 11a. For example, the second contact may be made multiple times within these time periods, for example, one or more times, two or more times, three or more times, four or more times, or five or more times. Further, for example, the first contact may be carried out within 1 hour, within 2 hours, within 3 hours, within 6 hours, within 12 hours, or within 24 hours after the first time of the contacting as a further contact using the first lipid nanoparticles 11a.

Here, the expression “appropriate affinity” means, for example, when referring to the target cells in the first state, that the affinity is higher than that of similar introduction carriers by general design under normal and/or general contacting conditions with the target cells in the first state. Such appropriate affinity can be achieved by adjusting the lipid composition of the lipid nanoparticles, as will be described in more detail later.

Here, the term “target cell” refers to cells to which the reprogramming factor is to be applied, and may be any general type of fibroblast. The organ from which the fibroblast is derived is not particularly limited. Further, examples of the fibroblasts include human fibroblasts, rat fibroblasts, feline fibroblasts, and canine fibroblasts, but the type is not limited to these. The fibroblasts can be selected according to the purpose of use. For example, for use in treatment, it is sufficient to use fibroblasts collected in advance from a patient to be treated. Note that fibroblasts are cells that are relatively easy to obtain. Therefore, compared to other cells, the technology for reprogramming of fibroblasts to create induced pluripotent stem cells or iPS cells is extremely useful over a wide range of fields, including medicine, esthetics, and basic research.

Here, the term “reprogramming factor” should sufficiently be a factor that can reprogram the DNA information in the nucleus when it is encapsulated in lipid nanoparticles and introduced into cells. For example, the reprogramming factor is mRNA, in which case, it is introduced into the cytoplasm of the reprogramming factor, it remain in the cytoplasm, and is translated into transcription factors, which is a protein as an reprogramming substance, and reprogram the DNA in the nucleus. The type of number of reprogramming factors may be one or more than two, and the necessary reprogramming factor may be introduced using the first lipid nanoparticles or the second lipid nanoparticles according to the state of the cells and the timing of administration. Examples of the reprogramming factor include, but are not limited to, messenger RNAs (mRNAs) of Oct3/4, Sox2, Klf4, cMyc, Nanog, and Lin28.

As described above, the lipid nanoparticles can be liposomes. They can be particles of lipid membranes that encapsulate cores of the aqueous solution, such as particles of lipid bilayer membranes. For the liposomes, any liposomes known themselves can be utilized. For example, the lipid composition which forms the liposomes can contain the first lipid (FFT-10) of Formula (I) and/or the second lipid (FFT-20) of Formula (II) as its components. These lipids are biodegradable lipids. By adjusting the lipid composition of the lipid nanoparticles using these lipids, appropriate affinity can be achieved.

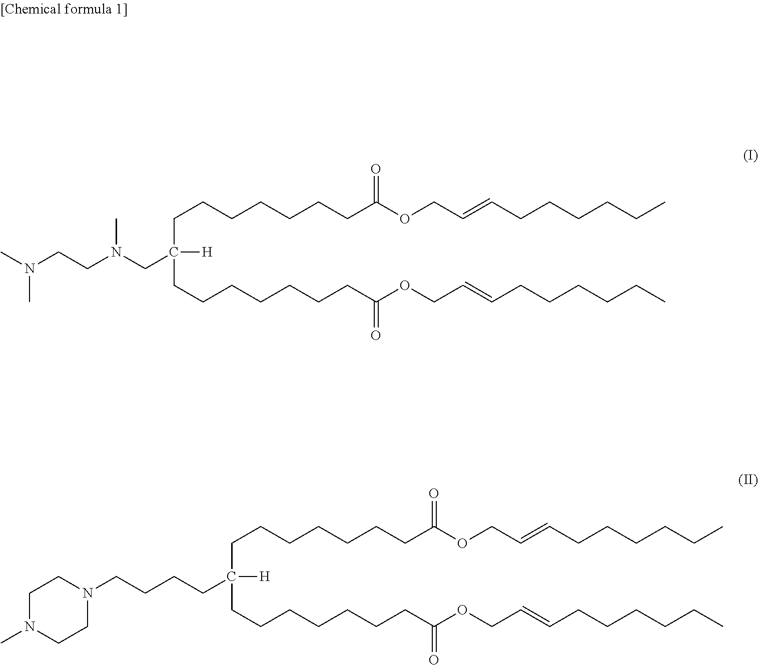

The lipid nanoparticles may as well contain further lipids in addition to the first lipids and second lipids described above. In the composition of the lipid molecular material that composes the lipid nanoparticles, a fraction constituted by the first lipids and second lipids is referred to as a “first fraction” hereinafter. Further, a fraction composed of lipid molecular materials other than those of the first lipids and the second lipids is referred to as a “second fraction” hereinafter. The lipids contained in the second fraction are collectively referred to as “third lipids” hereinafter.

The terms “first fraction” and “second fraction” express the type of composition of the components of the lipid nanoparticles, and do not indicate the physical location of the lipids contained therein. For example, the components of the first fraction and the second fraction do not need to be in separate groups within the lipid nanoparticles, and the lipids contained in the first fraction and the lipids contained in the second fraction can exist in a mixed state. The composition ratio of the first fraction to the total lipid material that makes up the lipid nanoparticles can be, for example, 30% to 70%, 30% to 65%, etc.

In other words, the total content of FFT-10 and/or FFT-20 can be, for example, 30% to 70% or 30% to 65% as a composition ratio of the lipid nanoparticles. In this context, percentages are expressed in mol % unless otherwise stated.

The average particle diameter of the lipid nanoparticles can be changed according to the usage, but for example, it can be adjusted to about 50 nm to about 300 nm. For example, it may as well be about 70 nm to about 100 nm.

The type of the third lipids contained in the second fraction of the lipid nanoparticles is not particularly limited, but for example, the second fraction contains base lipids. As the base lipid, for example, lipids that are a main component of a biological membrane can be used. Examples of the base lipids are a phospholipid and a sphingolipid, such as diacyl phosphatidyl choline, diacyl phosphatidyl ethanolamine, ceramide, sphingomyelin, dihydrosphingomyelin, cephalin, or cerebroside, or a combination of any of these.

For example, as base lipids, 1,2-dioleoyl-sn-glycero-3-phosphoethanolamine (DOPE), 1,2-stearoyl-sn-glycero-3-phosphoethanolamine (DSPE), 1,2-dipalmitoyl-sn-glycero-3-phosphatidylcholine (DPPC), 1-palmitoyl-2-oleoyl-sn-glycero-3-phosphatidylcholine (POPC), 1,2-di-O-octadecyl-3-trimethylammonium propane (DOTMA), 1,2-dioleoyl-3-dimethylammonium propane (DODAP), 1,2-dimyristoyl-3-dimethylammonium propane (14:0 DAP), 1,2-dipalmitoyl-3-dimethylammonium propane (16:0 DAP), 1,2-distearoyl-3-dimethylammonium propane (18:0 DAP), N-(4-Carboxybenzyl)-N,N-dimethyl-2,3-bis(oleoyloxy)propane (DOBAQ), 1,2-dioleoyl-3-trimethylammonium propane (DOTAP), 1,2-dioleoyl-sn-glycero-3-phosphocholine (DOPC), 1,2-Dilinoleoyl-sn-glycero-3-phosphocholine (DLPC), or 1,2-Dioleoyl-sn-glycero-3-phospho-L-serine (DOPS), or a combination of any of these can be used. As the base lipids, cationic lipids or neutral lipids should preferably be used, and the acid dissociation constant of the lipid nanoparticles can be adjusted by the content of the lipids. It is preferable to use DOTAP as the cationic lipids and DOPE as the neutral lipids.

It is also preferable that the second fraction should contain lipids that prevent aggregation of the lipid nanoparticles. Examples of the lipids that prevent aggregation can further include PEG-modified lipids, such as polyethylene glycol (PEG) dimyristoyl glycerol (DMG-PEG), polyamide oligomers derived from omega-amino (oligo ethylene glycol) alkanoate monomer (U.S. Pat. No. 6,320,017 B) and monosialogangliosides.

The second fraction may further contain lipids for adjusting toxicity, which have relatively low toxicity; lipids with functional groups that bind ligands to lipid nanoparticles; and lipids for inhibiting the leakage of internal contents such as sterols, for example, cholesterol. In particular, it is preferable to contain cholesterol.

The type and composition of the lipids used in the second fraction may be selected appropriately in consideration of the acid dissociation constant (pKa) of the lipid nanoparticles of the object, the particle diameter of the lipid nanoparticles, the type of reprogramming factor to be encapsulated, or the stability of the particles in cells or the like.

For example, the second fraction is preferable because it has particularly high efficiency in delivering the reprogramming factor, when it contains DOPE, DOTAP, cholesterol, and DMG-PEG.

In addition to the reprogramming factors, other components may be further encapsulated in the lipid nanoparticles as needed. Such further components include, for example, a pH adjuster, an osmotic pressure adjuster, and a gene activator. The pH adjuster may be an organic acid such as citric acid or a salt thereof. The osmotic pressure adjuster may be a sugar or amino acid. Here, the gene activator can be any substance that promotes or supports the activity of the reprogramming factor when the reprogramming factor is a gene.

The lipid nanoparticles that encapsulate the reprogramming factors and other substances as needed can be produced, for example, using known methods used for encapsulating small molecules in lipid nanoparticles, such as the Bangham method, organic solvent extraction method, surfactant removal method, or freeze-thaw method. For example, a lipid mixture obtained by adding a desired ratio of lipid nanoparticle material to an organic solvent such as alcohol, and an aqueous buffer solution containing the component to be encapsulated, such as reprogramming factors, are prepared, and the aqueous buffer solution is added to the lipid mixture. By agitating and suspending the resulting mixture, lipid nanoparticles that encapsulate the reprogramming factors, etc., are formed. The lipid nanoparticles obtained in this way are an example of liposomes.

For example, the lipid composition of the first lipid nanoparticles may contain FFT-10, DOPE, DOTAP, cholesterol, and DMG-PEG, and the ratio thereof in molar percentage may be in a range of: FFT T-10:DOPE:DOTAP:cholesterol:DMG-PEG=50 to 65:0:5 to 25:15 to 35:1 to 15. Or, for example, the lipid composition of the first lipid nanoparticles may contain FFT-10, DOPE, DOTAP, cholesterol, and DMG-PEG, and the ratio thereof in molar percentage may be in a range of: FFT-10:DOPE:DOTAP:cholesterol:DMG-PEG=53 to 63:0:9 to 21:19 to 29:1 to 7. Further, for example, the lipid composition of the second lipid nanoparticles may contain FFT-20, DOPE, DOTAP, cholesterol, and DMG-PEG, and the ratio thereof in molar percentage may be in a range of: be FFT T-20:DOPE:DOTAP:cholesterol:DMG-PEG=20 to 40:1 to 15:1 to 15:40 to 65:1 to 15. Or, for example, the lipid composition of the second lipid nanoparticles may contain FFT-20, DOPE, DOTAP, cholesterol, and DMG-PEG, and the ratio thereof in molar percentage may be in a range of: FFT-20:DOPE:DOTAP:cholesterol:DMG-PEG=26 to 37:1 to 9:4 to 14:46 to 61:1 to 8. The values of these ratios are selected so that the total of the lipid compositions of the first lipid nanoparticle and the second lipid nanoparticle is 100. Furthermore, for example, the lipid composition of the first lipid nanoparticles may contain FFT-10, DOPE, DOTAP, cholesterol, and DMG-PEG, and the ratio thereof in molar percentage may be 58:0:16:24:2. The lipid composition of the first lipid nanoparticles may contain FFT-10, DOPE, DOTAP, cholesterol, and DMG-PEG, and the lipid component ratio of the lipid nanoparticles may be FFT-10:DOPE:DOTAP:cholesterol:DMG-PEG=58.3:0:16.5:23.6:1.6 in molar percentage. For example, the lipid composition of the second lipid nanoparticles may contain FFT-20, DOPE, DOTAP, cholesterol, and DMG-PEG, and the ratio thereof in molar percentage may be 32:5:9:51:3. The lipid composition of the second lipid nanoparticles may contain FFT-20, DOPE, DOTAP, cholesterol, and DMG-PEG, and the lipid component ratio of the lipid nanoparticles may be FFT-20:DOPE:DOTAP:cholesterol:DMG-PEG=31.7:4.5:9.0:51.4:3.4 in molar percentage.

According to the method of the embodiment, it is possible to continuously introduce the reprogramming factors while changing the composition of the liposome for introduction along with the progression of the reprogramming process of the target cell. Further, with the change in the composition of the liposome, it is possible to maintain a certain amount or more of the reprogramming factors in the target cells at both the first half stage and the second half stage of the reprogramming process. When reprogramming factors are introduced into the initial material, fibroblasts, to produce iPS cells, the incomplete reprogramming is prevented. Thus, it is possible to maintain a sufficient amount of reprogramming factors in the cells for a certain period of time (for example, at least 4 days).

### Second Embodiment

An introduction carrier set according to the second embodiment will be explained with reference to FIG. 1. An introduction carrier set 2001 includes the first introduction carrier 101a and the second introduction carrier 201b. The first introduction carrier 101a includes the lipid nanoparticles 11a (for example, liposomes 11a) and the reprogramming factors 12a encapsulated therein. The lipid nanoparticles 11a have an affinity appropriate for fibroblasts as the initial material.

The second introduction carrier 201b includes the lipid nanoparticles 11b (for example, liposomes 11b) and the reprogramming factors 12a encapsulated therein. The lipid nanoparticles 11b have an affinity appropriate for fibroblasts in which reprogramming has been initiated. The introduction carrier set 2001 can maintain and/or enhance reprogramming by repeatedly introducing the reprogramming factors using two types of lipid nanoparticles having different component ratios. With this configuration, it is possible to efficiently perform reprogramming, and to obtain homogeneous reprogrammed cells and iPS cells.

### Third Embodiment

As described above, the introduction carrier set according to the second embodiment may be provided in a form that can be used immediately for the desired target cells, but it may also be provided in a form that can be adjusted by the user of the above-described introduction carrier set at the time of use. The third embodiment is an introduction carrier production kit provided in such a form.

The kit comprises, for example, a reprogramming factor;
materials for first lipid nanoparticles for encapsulating the reprogramming factor;
materials for second lipid nanoparticles for encapsulating the second reprogramming factor; and
the first lipid nanoparticles have a lipid composition designed to exhibit an affinity appropriate for fibroblasts as an initial material, and
the second lipid nanoparticle have a lipid composition different from that of the first lipid nanoparticles, which is designed to have an appropriate affinity for fibroblasts in a state in which reprogramming is initiated.

The materials for the first and second lipid nanoparticles and the reprogramming factors may be provided in a container in an appropriate state so that each of these is stably provided as a substance. Here, the reprogramming factors are initialization factors each to be encapsulated in the first and second lipid nanoparticle materials, respectively. Further, the kit may also include an instruction for preparing the introduction carrier for the user to properly manufacture the carrier, for example, a manufacturing manual.

It is possible to maintain and/or enhance reprogramming by repeatedly introducing reprogramming factors using two types of lipid nanoparticles with different component ratios that have been prepared using an introduction carrier production kit. Further, thereby, it is possible to perform reprogramming at high efficiency, and also possible to obtain homogeneous reprogrammed cells and iPS cells.

### EXAMPLES

Examples of the preparation and use of lipid nanoparticles as a form of lipid nanoparticles of the embodiments will be described.

### Experiment 1: Preparation of Lipid Nanoparticles Encapsulating Initiation Factor Group

As the nucleic acids to be encapsulated in the lipid nanoparticles, the messenger RNAs of 6 types of initialization factors (Oct3/4, Sox2, Klf4, cMyc, Nanog, and Lin28) were used. The nucleic acids were suspended in 10 mM HEPES (pH 7.3) to obtain a nucleic acid solution.

FFT-10, FFT-20, DOPE, DOTAP, cholesterol, and DMG-PEG were dissolved in ethanol at the lipid composition ratios shown in Table 1 to obtain lipid solutions. The lipid solutions and the above-described nucleic acid solutions were mixed using a microflow chip and syringe pump. After diluting the mixed solution 10-fold with 10 mM HEPES (pH 7.3), it was concentrated using an ultrafiltration filter (Amicon Ultra 0.5 Ultracel-50, Merck) to obtain lipid nanoparticles. The lipid nanoparticles thus obtained were used in the experiments that followed, that is, Example 1 as the first lipid nanoparticles and Example 2 as the second lipid nanoparticles.

TABLE 1

| | Composition ratio of |
|---|---|
| | lipid particle prepared (mol %) |
| | (FFT-10:FFT-20:DOPE:DOTAP:Cholesterol:DMG-PEG) |
| Example | 58:0:0:16:24:2 |
| 1 | |
| Example | 0:32:5:9:51:3 |
| 2 | |

### Experiment 2: Measurement of Concentration of Encapsulated Nucleic Acid

The concentration of the encapsulated nucleic acid in the lipid nanoparticles prepared in each case of Experiment 1 was measured using QuantiFluor RNA system (Promega). After adding Triton X-100 (0.1 v/v %, Eastman Kodak Company) to the lipid nanoparticle solution and mixing it with a vortex mixer, the mixture was left to stand at room temperature for 30 minutes. After adding an equal amount of QuantiFluor solution and let it stand at room temperature for 10 minutes, the fluorescence intensity was measured using a single-tube fluorescence measurement device, Quantus Fluorometer (Promega). The concentration of the nucleic acid encapsulated in the lipid nanoparticles was calculated by subtracting the fluorescence intensity of the sample obtained without adding Triton X-100 from the fluorescence intensity of the sample with Triton X-100 added. TABLE 2 shows the concentration of the nucleic acid encapsulated in the lipid nanoparticles in each case.

TABLE 2

| | Concentration of |
|---|---|
| | encapsulated nucleic acid |
| | (ng/μL) |
| Example 1 | 67.7 |
| Example 2 | 98.6 |

### Experiment 3: Cell Initialization Test

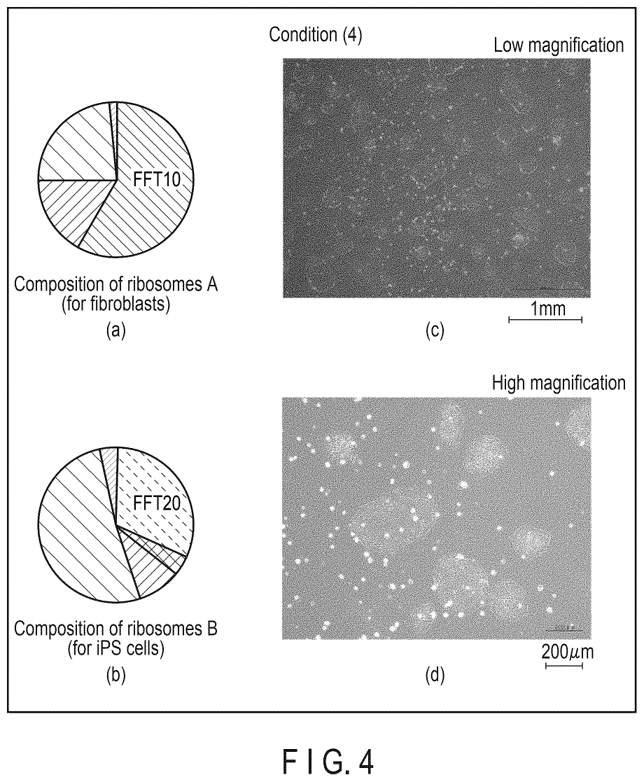

Human fibroblasts (Fibroblast, KURABO, Lot. 06444) cultured in Dulbecco's Modified Eagle Medium (DMEM, Gibco) were centrifuged and collected. Then, the resultants were seeded on a 24-well culture plate at 4×104 cells/well (each of the well contains 300 μL of DMEM (with the addition of 10% fetal bovine serum)), and each of the lipid nanoparticles prepared in Experiment 1 was added under the conditions indicated in TBLAE 3. In each case, the culture plate was placed in an incubator and the cells were cultured at 37° C. in an atmosphere of 5% CO₂. Every 3 to 4 days, the cells were cultured while adding AK03N medium (with the addition of Rock Inhibitor Y27632), and whether or not iPS cell colonies were formed was observed under an optical microscope. FIG. 4 shows images of the lipid composition of the liposomes used in Example 1 and Example 2 (part (a) of FIG. 4 and part (b) of FIG. 4), and images of the cells observed under an optical microscope after culturing for 4 weeks while sequentially adding the lipid nanoparticles of Example 1 and Example 2 under Condition 3 (part (c) of FIG. 4 and part (d) of FIG. 4). In any of the observed cell colonies, slender and multi-directional protrusions, which are characteristic of fibroblasts, were not observed, but characteristics of iPS cell colonies, which are arranged to be round paving stone, were observed. Based on the morphological characteristics, it was confirmed that iPS cells were produced from fibroblasts under Condition 3.

As a comparative experiment, messenger RNAs for six reprogramming factors (Oct3/4, Sox2, Klf4, cMyc, Nanog, and Lin28) were introduced using a lipofection reagent (LipoFectamine RNAiMAX, Thermo Fisher Scientific) and observed under a microscope.

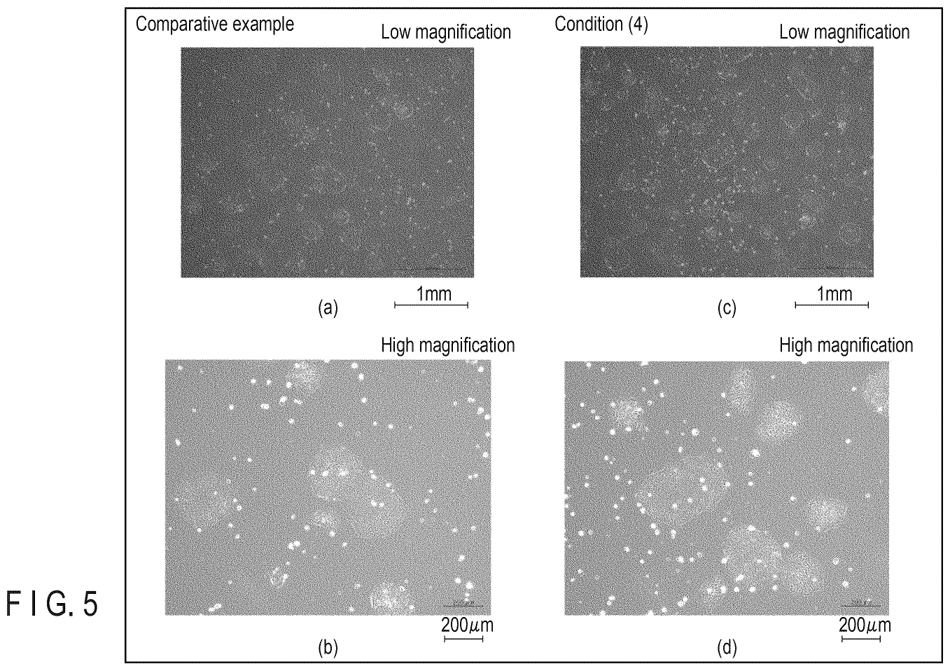

The results of Examples 1 and 2 and the comparative experiment conducted using the lipofection method are shown in FIG. 5, and the conditions used and the experimental results are summarized in Table 3.

TABLE 3

| | Day 0 | Day 1 | Day 2 | Day 3 | Day 4 |
|---|---|---|---|---|---|
| Condition | Example | — | — | — | — |
| 1 | 1 | | | | |
| | 400 ng/ | | | | |
| | well | | | | |
| Condition | Example | Example | Example | Example | — |
| 2 | 1 | 1 | 1 | 1 | |
| | 400 ng/ | 400 ng/ | 400 ng/ | 400 ng/ | |
| | well | well | well | well | |
| Condition | Example | — | Example | — | Example |
| 3 | 1 | | 2 | | 2 |
| | 400 ng/ | | 400 ng/ | | 400 ng/ |
| | well | | well | | well |
| Comparative | Hit1 | Hit2 | Hit3 | Hit4 | — |
| test | 400 ng/ | 400 ng/ | 400 ng/ | 400 ng/ | |
| | well | well | well | well | |

Of the three conditions that were carried out by changing the type of lipid nanoparticles added and the timing of the addition, Condition 3, in which the lipid nanoparticles of Example 1 were added on Day 0 and the lipid nanoparticles of Example 2 were added on Day 2 and Day 4, was observed as that iPS cell colonies were formed after about four weeks. The morphology of the same colonies under the conditions according to the embodiment (part (c) of FIG. 5 and part (d) of FIG. 5) was observed to have such a tendency to be equivalent or better than that of the comparative experiment conducted as a comparison, that is, the iPS cell colonies formed when the lipofection reagent was administered four times continuously from Day 0 to Day 3 (part (a) of FIG. 5 and (b) of FIG. 5). In the case of Condition 1 (in which the lipid nanoparticles of Example 1 were added on Day 0) and Condition 2 (in which the lipid nanoparticles of Example 1 was add four times in succession from Day 0 to Day 3), which correspond to the conditions where only lipid nanoparticles were used, the sufficient formation of the target iPS cell colonies was not observed.

### Experiment 4: Study on Timing of Addition of Initialization Factors

As in the cases of Experiments 1 to 3, experiments were conducted using lipid nanoparticles by changing the timing of adding the reprogramming factors and the formation of iPS cell colonies was confirmed. The results are shown in TABLE 4.

TABLE 4
Timing of addition of liposomes, results of production of iPS cells

| | | | | | | iPS |
|---|---|---|---|---|---|---|
| | Day 0 | Day 1 | Day 2 | Day 3 | Day 4 | formed |
| Condition | A | — | — | — | — | X |
| 1 | | | | | | |
| Condition | A | A | A | A | — | X |
| 2 | | | | | | |
| Condition | A | — | B | — | B | ◯ |
| 3 | | | | | | |

The results are described as A for the addition of the lipid nanoparticles in Example 1 and B for the addition of the lipid nanoparticles in Example 2. For Condition 1, A was added one time on Day 0, for Condition 2, A was added four times from Day 0 to Day 3, and for Condition 3, A was added one time on Day 0, B was added one time on Day 2, and B was added one time on Day 4, for a total of three additions. For Condition 3, it was observed that iPS cell colonies were well formed. In contrast, for Conditions 1 and 2, the formation of iPS cell colonies was not observed.

From the above results, it was confirmed that it is possible to initialize fibroblasts and thereby produce iPS cells using the lipid nanoparticles and the methods according to the embodiments.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The followings are descriptions of examples of further embodiments.

(1) A method of producing an induced pluripotent stem cell, comprising:
bringing a first lipid nanoparticle encapsulating a reprogramming factor into contact at least once with a fibroblast; and
bringing, after at least once of the contact, a second lipid nanoparticle encapsulating the reprogramming factor and having a lipid component composition ratio different from that of the first lipid nanoparticle into contact at least once with the fibroblast; and
obtaining an induced pluripotent stem cell.

(2) The method according to clause (1), wherein the first and second lipid nanoparticles are lipid nanoparticles composed of lipid components including FFT-10 and/or FFT-20, which are biodegradable lipids.

(3) The method according to clause (1) or (2), wherein the first and second lipid nanoparticles have a ratio of FFT-10 and/or FFT-20 of 30% or more.

(4) The method according to any one of clauses (1) to (3), wherein the lipid component of the first lipid nanoparticle has a higher content of FFT-10 than that of the second lipid nanoparticle.

(5) The method according to any one of clauses (1) to (5), wherein the lipid composition of the first lipid nanoparticle contains FFT-10, DOPE, DOTAP, cholesterol, and DMG-PEG, and a ratio thereof in percentage by molar is in a range of FFT-10:DOPE:DOTAP:cholesterol:DMG-PEG=50 to 65:0:5 to 25:15 to 35:1 to 15, where respective values are selected so that a total thereof is 100, and
the lipid composition of the second lipid nanoparticle contains FFT-20, DOPE, DOTAP, cholesterol, and DMG-PEG, and a ratio thereof in percentage by molar is in a range of FFT-20:DOPE:DOTAP cholesterol:DMG-PEG=20 to 40:1 to 15:1 to 15:40 to 65:1 to 15, where respective values are selected so that a total thereof is 100.

(6) The method according to any one of clauses (1) to (5), wherein the lipid composition of the first lipid nanoparticle contains FFT-10, DOPE, DOTAP, cholesterol, and DMG-PEG, and a ratio thereof in percentage by molar is in a range of FFT-10:DOPE:DOTAP:cholesterol:DMG-PEG=53 to 63:0:9 to 21:19 to 29:1 to 7, where respective values are selected so that a total thereof is 100, and
the lipid composition of the second lipid nanoparticle contains FFT-20, DOPE, DOTAP, cholesterol, and DMG-PEG, and a ratio thereof in percentage by molar is in a range of FFT-20:DOPE:DOTAP:cholesterol:DMG-PEG=26 to 37:1 to 9:4 to 14:46 to 61:1 to 8, where respective values are selected so that a total thereof is 100.

(7) The method according to any one of clauses (1) to (5), wherein
the lipid composition of the first lipid nanoparticle contain FFT-10, DOPE, DOTAP, cholesterol, and DMG-PEG, and a constitutional ratio of the lipid component in percentage by molar is: FFT-10:DOPE:DOTAP:cholesterol:DMG-PEG=58.3:0:16.5:23.6:1.6, and
the lipid composition of the second lipid nanoparticle contain FFT-20, DOPE, DOTAP, cholesterol, and DMG-PEG, and a constitutional ratio of the lipid component in percentage by molar is: FFT-20:DOPE:DOTAP:cholesterol:DMG-PEG=31.7:4.5:9.0:51.4:3.4.

(8) An introduction carrier set for use in any one of the method according to any one of clauses (1) to (7), comprising:
a first lipid nanoparticle encapsulating a reprogramming factor and having a lipid composition designed to show an affinity appropriate for a fibroblast in a state of an initial material;
a second lipid nanoparticle encapsulating the reprogramming factor and having a lipid composition different from that of the first lipid nanoparticle, and designed to show an affinity appropriate for a fibroblast in which reprogramming is initiated.

(9) A kit for manufacturing an introduction carrier set for use in the method according to any one of clauses (1) to (7), comprising:
a material for first lipid nanoparticle encapsulating a reprogramming factor and having a lipid composition designed to show an affinity appropriate for a fibroblast in a state of an initial material;
a material for second lipid nanoparticle encapsulating the reprogramming initialization factor and having a lipid composition different from that of the first lipid nanoparticle, and designed to show an affinity appropriate for a fibroblast in which reprogramming initialization is initiated; and
reprogramming factors to be encapsulated in the first lipid nanoparticle and the second lipid nanoparticle, respectively.

(10) A kit according to clause (9), further comprising a manufacture manual.

What is claimed is:

1. A method of producing an induced pluripotent stem cell, comprising:
bringing a first lipid nanoparticle encapsulating an reprogramming factor into contact at least once with a fibroblast; and
bringing, after at least once of the contact, a second lipid nanoparticle encapsulating the reprogramming factor and having a lipid component composition ratio different from that of the first lipid nanoparticle into contact at least once with the fibroblast; and
obtaining an induced pluripotent stem cell.

2. The method of claim 1, wherein the first and second lipid nanoparticles are lipid nanoparticles composed of lipid components including FFT-10 and/or FFT-20, which are biodegradable lipids.

3. The method of claim 1, wherein the first and second lipid nanoparticles have a ratio of FFT-10 and/or FFT-20 of 30% or more.

4. The method of claim 1, wherein the lipid component of the first lipid nanoparticle has a higher content of FFT-10 than that of the second lipid nanoparticle.

5. The method of claim 1, wherein the lipid composition of the first lipid nanoparticle contains FFT-10, DOPE, DOTAP, cholesterol, and DMG-PEG, and a ratio thereof in percentage by molar is in a range of: FFT-10:DOPE:DOTAP:cholesterol DMG-PEG=50 to 65:0:5 to 25:15 to 35:1 to 15, where respective values are selected so that a total thereof is 100, and
the lipid composition of the second lipid nanoparticle contains FFT-20, DOPE, DOTAP, cholesterol, and DMG-PEG, and a ratio thereof in percentage by molar is in a range of: FFT-20:DOPE:DOTAP cholesterol:DMG-PEG=20 to 40:1 to 15:1 to 15:40 to 65:1 to 15, where respective values are selected so that a total thereof is 100.

6. The method of claim 1, wherein the lipid composition of the first lipid nanoparticle contains FFT-10, DOPE, DOTAP, cholesterol, and DMG-PEG, and a ratio thereof in percentage by molar is in a range of: FFT-10:DOPE:DOTAP:cholesterol DMG-PEG=53 to 63:0:9 to 21:19 to 29:1 to 7, where respective values are selected so that a total thereof is 100, and
the lipid composition of the second lipid nanoparticle contains FFT-20, DOPE, DOTAP, cholesterol, and DMG-PEG, and a ratio thereof in percentage by molar is in a range of: FFT-20:DOPE:DOTAP:cholesterol:DMG-PEG=26 to 37:1 to 9:4 to 14:46 to 61:1 to 8, where respective values are selected so that a total thereof is 100.

7. The method of claim 1, wherein
the lipid composition of the first lipid nanoparticle contain FFT-10, DOPE, DOTAP, cholesterol, and DMG-PEG, and a constitutional ratio of the lipid component in percentage by molar is: FFT-10:DOPE:DOTAP:cholesterol:DMG-PEG=58.3 0:16.5:23.6:1.6, and
the lipid composition of the second lipid nanoparticle contain FFT-20, DOPE, DOTAP, cholesterol, and DMG-PEG, and a constitutional ratio of the lipid component in percentage by molar is: FFT-20:DOPE:DOTAP:cholesterol:DMG-PEG=31.7:4.5:9.0:51.4:3.4.

8. An introduction carrier set for use in the method of claim 1, comprising:
a first lipid nanoparticle encapsulating a reprogramming factor and having a lipid composition designed to show an affinity appropriate for a fibroblast in a state of an initial material;
a second lipid nanoparticle encapsulating the reprogramming factor and having a lipid composition different from that of the first lipid nanoparticle, and designed to show an affinity appropriate for a fibroblast in which reprogramming is initiated.

9. A kit for manufacturing an introduction carrier set for use in the method of claim 1, comprising:
a material for first lipid nanoparticle encapsulating a reprogramming factor and having a lipid composition designed to show an affinity appropriate for a fibroblast in a state of an initial material;
a material for second lipid nanoparticle encapsulating the reprogramming factor and having a lipid composition different from that of the first lipid nanoparticle, and designed to show an affinity appropriate for a fibroblast in which reprogramming is initiated; and
reprogramming factors to be encapsulated in the first lipid nanoparticle and the second lipid nanoparticle, respectively.

10. The kit of claim 9, further comprising a manufacture manual.
